(19) United States
(12) Patent Application Publication
(10) Pub. No.: US 2018/0264916 A1
(43) Pub. Date: Sep. 20, 2018

(54) Medicated Vapor Dispensing Device

(71) Applicants: Duong Hang, Las Vegas, NV (US); Danielle Ayala, Rohnert Park, CA (US)

(72) Inventors: Duong Hang, Las Vegas, NV (US); Danielle Ayala, Rohnert Park, CA (US)

(21) Appl. No.: 15/459,325

(22) Filed: Mar. 15, 2017

(51) Int. Cl. B60H 3/00; A61M 11/02

(52) U.S. Cl. CPC B60H 3/0028; A61M 2209/082; A61M 11/02

(57) ABSTRACT

A medicated vapor dispensing device for a vehicle includes a housing that defines an internal space. A plurality of openings is positioned through a back of the housing and a plurality of apertures is positioned through a front of the housing. A coupler is coupled to the back of the housing. The coupler is configured to couple the housing to a vent of the vehicle positioning the plurality of openings to admit air exiting the vent into the internal space. Each of a plurality of pads is selectively impregnated with a substrate that is configured to vaporize from the pad. A slot that is positioned through a top of the housing is configured to selectively insert a respective pad into the internal space. The respective pad is configured to emit the substrate into air passing through the internal space into an interior space of the vehicle.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to vapor dispensing devices and more particularly pertains to a new vapor dispensing device for a vehicle.

### BRIEF SUMMARY OF THE INVENTION

An embodiment of the disclosure meets the needs presented above by generally comprising a housing that defines an internal space. A plurality of openings is positioned through a back of the housing and a plurality of apertures is positioned through a front of the housing. A coupler is coupled to the back of the housing. The coupler is configured to couple the housing to a vent of a vehicle positioning the plurality of openings to admit air exiting the vent into the internal space. Each of a plurality of pads is selectively impregnated with a substrate that is configured to vaporize from the pad. A slot that is positioned through a top of the housing is configured to selectively insert a respective pad into the internal space. The respective pad is configured to emit the substrate into air passing through the internal space into an interior space of the vehicle.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric perspective view of a medicated vapor dispensing device according to an embodiment of the disclosure.

FIG. 2 is a back view of an embodiment of the disclosure.

FIG. 3 is a side view of an embodiment of the disclosure.

FIG. 4 is a cross-sectional view of an embodiment of the disclosure.

FIG. 5 is an in-use view of an embodiment of the disclosure.

### DETAILED DESCRIPTION OF THE INVENTION

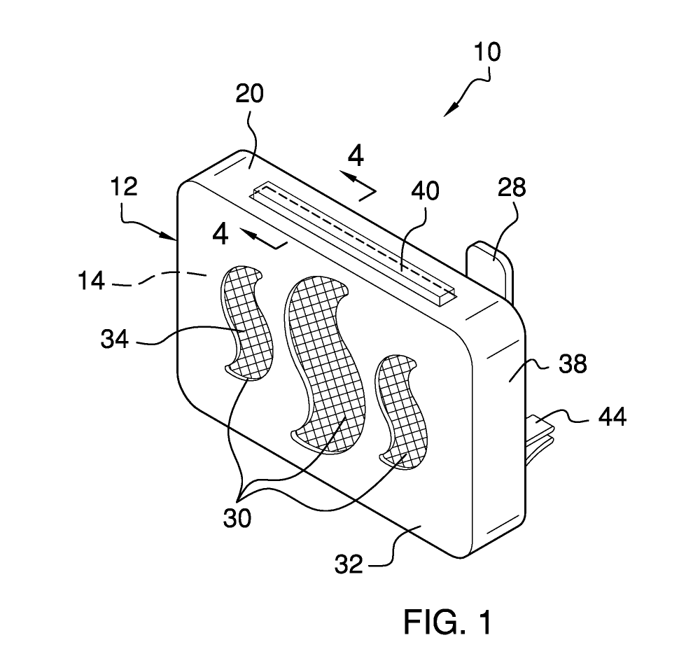

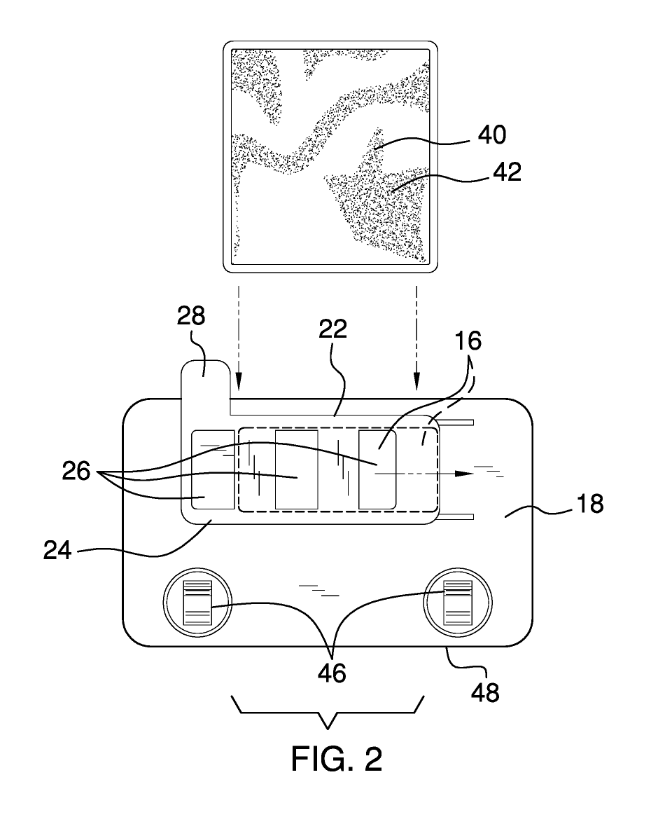

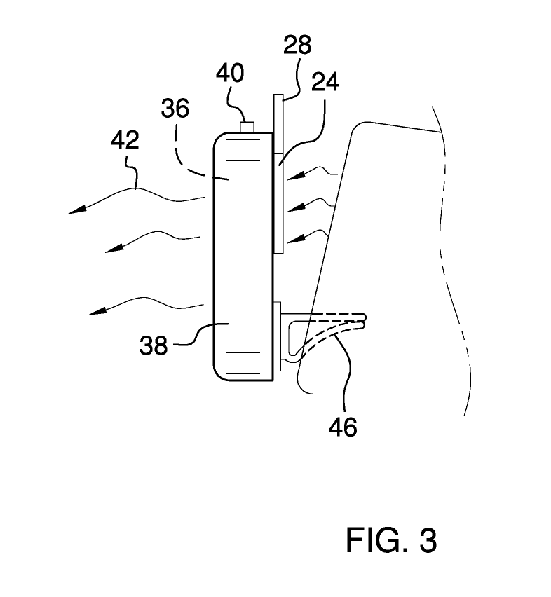

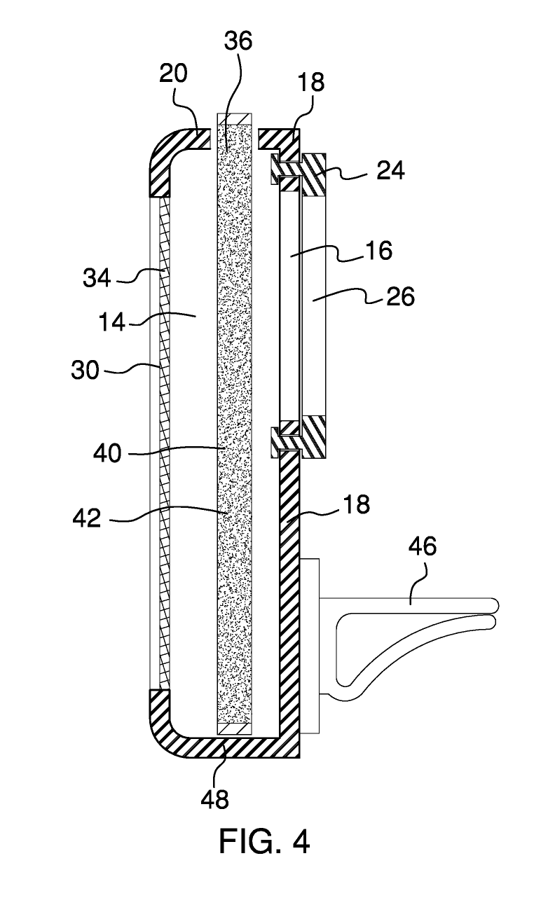

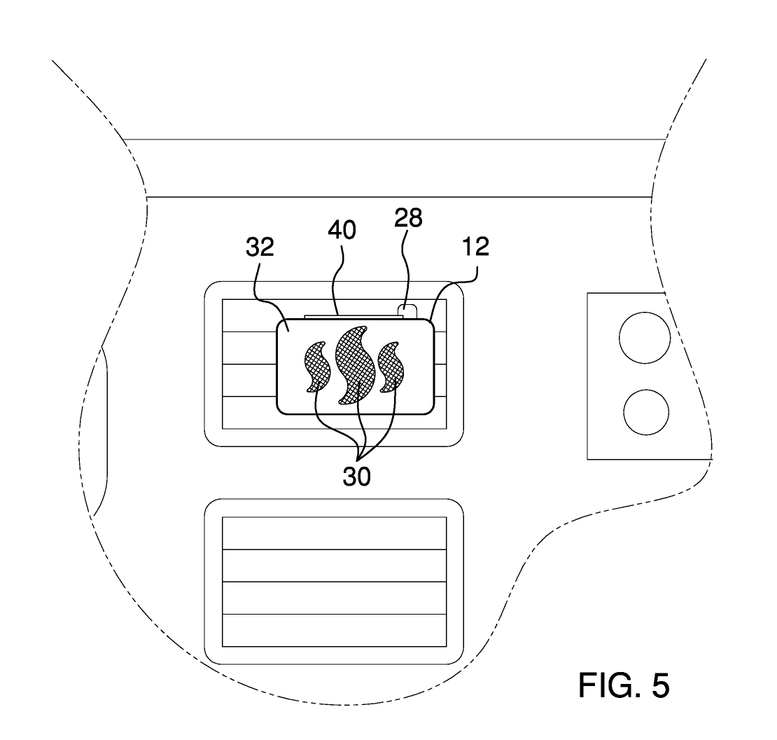

With reference now to the drawings, and in particular to FIGS. 1 through 5 thereof, a new vapor dispensing device embodying the principles and concepts of an embodiment of the disclosure and generally designated by the reference numeral 10 will be described.

As best illustrated in FIGS. 1 through 5, the medicated vapor dispensing device 10 generally comprises a housing 12 that defines an internal space 14. In one embodiment, the housing 12 is substantially rectangularly box shaped.

A plurality of openings 16 is positioned through a back 18 of the housing 12. The openings 16 are configured to admit air into the internal space 14. In one embodiment, the openings 16 are positioned proximate to a top 20 of the housing 12.

A dampener 22 is coupled to the housing 12 proximate to the openings 16. The dampener 22 is configured to adjustably control airflow through the openings 16 into the internal space 14. In one embodiment, the dampener 22 comprises a slat 24 that is slidably coupled to the back 18 of the housing 12. The slat 24 is positioned over the plurality of openings 16. A plurality of penetrations 26 is positioned through the slat 24. The penetrations 26 are substantially complementary to the openings 16. Each penetration 26 is selectively alignable with an associated opening 16. The associated opening 16 is selectively exposed through the penetration 26. The airflow through the openings 16 into the internal space 14 is controlled.

A tab 28 is coupled to and extends perpendicularly from the slat 24 past the top 20 of the housing 12. The tab 28 is configured to motivate the slat 24 relative to the openings 16 such that each penetration 26 is selectively alignable with the associated opening 16.

A plurality of apertures 30 is positioned through a front 32 of the housing 12. The apertures 30 are configured to release the air from the internal space 14 to an interior space of a vehicle. In one embodiment, each aperture 30 is S-shaped. In another embodiment, the plurality of apertures 30 comprises three apertures 30. In yet another embodiment, a screen 34 is coupled to the housing 12. The screen 34 is positioned to cover the plurality of apertures 30.

A slot 36 is positioned through the top 20 of the housing 12. In one embodiment, the slot 36 is substantially rectangularly shaped. In another embodiment, the slot 36 extends substantially between opposing sides 38 of the housing 12.

Each of a plurality of pads 40 is selectively impregnated with a substrate 42. The substrate 42 is configured to vaporize from the pad 40. The slot 36 is configured to selectively insert a respective pad 40 into the internal space 14. The respective pad 40 is configured to emit the substrate 42 into the air passing through the internal space 14. In one embodiment, the substrate 42 is medicinal. The substrate 42 is configured to treat a respiratory ailment, such as influenza, common cold, and allergy, of an occupant of the vehicle.

A coupler 44 is coupled to the back 18 of the housing 12. The coupler 44 is configured to couple the housing 12 to a vent of the vehicle, positioning the plurality of openings 16 to admit air that exits the vent into the internal space 14.

In one embodiment, the coupler 44 comprises a pair of clips 46. Each clip 46 is positioned proximate to a bottom 48 and a respective opposing side 38 of the housing 12. The clips 46 are configured to couple to a louver of the vent of the vehicle, positioning the plurality of openings 16 to admit the air that exits the vent into the internal space 14.

In use, the clips 46 are configured to couple to the louver of the vent of the vehicle, positioning the plurality of openings 16 to admit the air that exits the vent into the internal space 14. The tab 28 is configured to motivate the slat 24 relative to the openings 16 such that each penetration 26 is selectively alignable with the associated opening 16. The airflow through the openings 16 into the internal space 14 is controlled. The slot 36 is configured to selectively insert the respective pad 40 into the internal space 14. The respective pad 40 is configured to emit the substrate 42 into the air passing through the internal space 14. The apertures 30 are configured to release the air from the internal space 14 to the interior space of the vehicle. The substrate 42 is configured to treat the respiratory ailment, such as influenza, common cold and allergy, of the occupant of the vehicle.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. Medicated vapor dispensing device comprising:
a housing defining an internal space;
a plurality of openings positioned through a back of said housing;
a plurality of apertures positioned through a front of said housing;
a slot positioned through a top of said housing;
a plurality of pads, each said pad being selectively impregnated with a substrate, said substrate being configured for vaporizing from said pad, wherein said slot is positioned in said housing such that said slot is configured for selectively inserting a respective said pad into said internal space, such that said pad is configured for emitting said substrate into air passing through said internal space;
a coupler coupled to said back of said housing, said coupler being configured for coupling said housing to a vent of a vehicle; and
wherein said openings are positioned in said housing such that said openings are configured for admitting the air into said internal space, wherein said apertures are positioned in said housing such that said apertures are configured for releasing the air from said internal space to an interior space of the vehicle, wherein said slot is positioned in said housing such that said slot is configured for selectively inserting said respective said pad into said internal space, such that said respective said pad is configured for emitting said substrate into the air passing through said internal space, wherein said coupler is positioned on said housing such that said coupler is configured for coupling said housing to the vent positioning said plurality of openings for admitting air exiting the vent into said internal space.

2. The device of claim 1, further including said housing being substantially rectangularly box shaped.

3. The device of claim 1, further including said openings being positioned proximate to said top of said housing.

4. The device of claim 1, further including a dampener coupled to said housing proximate to said openings, said dampener being configured for adjustably controlling airflow through said openings into said internal space.

5. The device of claim 4, further including said dampener comprising:
a slat slidably coupled to said back of said housing such that said slat is positioned over said plurality of openings;
a plurality of penetrations positioned through said slat, said penetrations being substantially complementary to said openings; and
wherein each said penetration is selectively alignable with an associated said opening such that said associated said opening is selectively exposable through said penetration such that the airflow through said openings into said internal space is controllable.

6. The device of claim 5, further including a tab coupled to and extending perpendicularly from said slat past said top of said housing, wherein said tab is positioned on said slat such that said tab is configured for motivating said slat relative to said openings such that each said penetration is selectively alignable with said associated said opening.

7. The device of claim 1, further including each said aperture being S-shaped.

8. The device of claim 1, further including said plurality of apertures comprising three said apertures.

9. The device of claim 1, further including a screen coupled to said housing, said screen being positioned to cover said plurality of apertures.

10. The device of claim 1, further including said slot being substantially rectangularly shaped.

11. The device of claim 1, further including said slot extending substantially between opposing sides of said housing.

12. The device of claim 1, further including said substrate being medicinal such that said substrate is configured for treating a respiratory ailment, such as influenza, common cold and allergy, of an occupant of the vehicle.

13. The device of claim 1, further including said coupler comprising a pair of clips, each said clip being positioned proximate to a bottom and a respective opposing side of said housing, wherein said clips are positioned on said housing such that said clips are configured for coupling to a louver of the vent of the vehicle positioning said plurality of openings for admitting air exiting the vent into said internal space.

14. Medicated vapor dispensing device comprising:
a housing defining an internal space, said housing being substantially rectangularly box shaped;
a plurality of openings positioned through a back of said housing, wherein said openings are positioned in said housing such that said openings are configured for admitting air into said internal space, said openings being positioned proximate to a top of said housing;
a dampener coupled to said housing proximate to said openings, said dampener being configured for adjustably controlling airflow through said openings into said internal space, said dampener comprising:
a slat slidably coupled to said back of said housing such that said slat is positioned over said plurality of openings,
a plurality of penetrations positioned through said slat, said penetrations being substantially complementary to said openings, wherein each said penetration is selectively alignable with an associated said opening such that said associated said opening is selectively exposable through said penetration such that the airflow through said openings into said internal space is controllable, and
a tab coupled to and extending perpendicularly from said slat past said top of said housing, wherein said tab is positioned on said slat such that said tab is configured for motivating said slat relative to said openings such that each said penetration is selectively alignable with said associated said opening;
a plurality of apertures positioned through a front of said housing, wherein said apertures are positioned in said housing such that said apertures are configured for releasing the air from said internal space to an interior space of a vehicle, each said aperture being S-shaped, said plurality of apertures comprising three said apertures;
a screen coupled to said housing, said screen being positioned to cover said plurality of apertures;
a slot positioned through said top of said housing, said slot being substantially rectangularly shaped, said slot extending substantially between opposing sides of said housing;
a plurality of pads, each said pad being selectively impregnated with a substrate, said substrate being configured for vaporizing from said pad, wherein said slot is positioned in said housing such that said slot is configured for selectively inserting a respective said pad into said internal space, such that said respective said pad is configured for emitting said substrate into the air passing through said internal space, said substrate being medicinal such that said substrate is configured for treating a respiratory ailment, such as influenza, common cold and allergy, of an occupant of the vehicle;
a coupler coupled to said back of said housing, said coupler being configured for coupling said housing to a vent of the vehicle, wherein said coupler is positioned on said housing such that said coupler is configured for coupling said housing to the vent positioning said plurality of openings for admitting air exiting the vent into said internal space, said coupler comprising a pair of clips, each said clip being positioned proximate to a bottom and a respective said opposing side of said housing, wherein said clips are positioned on said housing such that said clips are configured for coupling to a louver of the vent of the vehicle positioning said plurality of openings for admitting the air exiting the vent into said internal space; and
wherein said clips are positioned on said housing such that said clips are configured for coupling to the louver of the vent of the vehicle positioning said plurality of openings for admitting the air exiting the vent into said internal space, wherein said tab is positioned on said slat such that said tab is configured for motivating said slat relative to said openings such that each said penetration is selectively alignable with said associated said opening such that the airflow through said openings into said internal space is controllable, wherein said slot is positioned in said housing such that said slot is configured for selectively inserting said respective said pad into said internal space, such that said respective said pad is configured for emitting said substrate into the air passing through said internal space, wherein said apertures are positioned in said housing such that said apertures are configured for releasing the air from said internal space to the interior space of the vehicle such that said substrate is configured for treating the respiratory ailment, such as influenza, common cold and allergy, of the occupant of the vehicle.
